(19) United States
(12) Patent Application Publication
(10) Pub. No.: US 2011/0207988 A1
(43) Pub. Date: Aug. 25, 2011

(54) Method, apparatus and computer program for non-invasive brain stimulation when target muscles are suitably active

(75) Inventors: Jarmo Ruohonen, Helsinki (FI); Henri Hannula, Helsinki (FI)

(73) Assignee: NEXSTIM OY, Helsinki (FI)

(21) Appl. No.: 13/126,493

(22) PCT Filed: Oct. 31, 2008
(86) PCT No.: PCT/FI2008/050620

§ 371 Date: Apr. 28, 2011

(51) Int. Cl. A61N 2/00

(52) U.S. Cl. 600/9

(57) ABSTRACT

The present invention introduces a magnetic stimulation method in which a desired biosignal value or a range of values for at least one target is determined. Upon stimulation, magnetic field pulses of short duration are applied to the brain and the biosignal, such as electromyograph (EMG), value (S) of each target, such as a muscle, is measured before each TMS pulse. The firing of a TMS pulse is automatically prevented if the corresponding measured biosignal value (S) is outside the predetermined limits.

### FIELD OF THE INVENTION

The present invention relates to stimulating biological tissue for medical purposes. In particular, the present invention relates to a method and apparatus for generating stimulating magnetic field pulses to the brain. To be precise, the present invention relates to what is stated in the preamble portion of the independent claims.

### BACKGROUND OF THE INVENTION

Biological tissue, such as the human brain, can be stimulated non-invasively for the purpose of providing information useful for diagnosis or treatment, or for the purpose of providing therapeutic effect. Using conventional techniques, it is possible to stimulate biological tissue by virtue of inducing an electric field in the tissue. The technique of magnetic stimulation accomplishes this by means of a changing magnetic field. Various apparatuses and methods for providing biological tissue with magnetic stimulation are disclosed, e.g., in publications:
U.S. Pat. No. 4,940,453
U.S. Pat. No. 5,766,124
U.S. Pat. No. 6,132,361 and
U.S. Pat. No. 6,086,525.

In these methods typically sinusoidally fluctuating and damped electric current pulses are selectively applied to a stimulator coil that is placed over the neurons to be stimulated. When the objective is to stimulate the brain, the method is called transcranial magnetic stimulation (TMS), which offers a risk- and pain-free method of stimulating the human brain. TMS is conventionally targeted over the areas of the brain controlling movements. This part of the brain is referred to as the motor cortex. Stimulation of the motor cortex triggers neuronal signals that travel from the stimulated cortex through pyramidal cell fibers and peripheral fibers, on to the muscles. A successfully transmitted neuronal signal results in a contraction of the muscle, which is seen as visible twitches. Muscular activity can also be detected as electrical signals from the muscles or the surface of the skin using electromyograph (EMG). Conventional techniques for combining evoked response and TMS measurements are disclosed in publication U.S. Pat. No. 4,940,453. Applications and technology have been covered extensively in several books, including the Oxford Handbook of Transcranial Stimulation, edited by E Wassermann et al., 2008 and the Magnetic Stimulation in Clinical Neurophysiology, edited by Hallett M, Chokrovery S, 2005, Elsevier.

In addition to EMG signals that are measured from the muscles, TMS stimulation results in other measurable changes elsewhere in the human body (i.e., biosignals). The most prominent detectable changes are in the brain's metabolic activity and in electrical signaling between the neurons. Metabolic changes can be detected using, for instance, functional MRI or Positron emission tomography or single-photon emission tomography (fMRI, PET and SPECT). Electrical changes can be detected using electoencephalography (EEG). Elsewhere in the body the effects of TMS stimulation are generally indirect, and detected, e.g., as change in ECG (electrocardiography).

Generally speaking, TMS stimulation results in various detectable biosignals in the human body. Frequently TMS experiments are conducted to study the linkage between the stimulated brain area and the detected change in a biosignal. In any such experiment it is fundamentally important to stabilize the state of the brain and the state of the “generator” of the biosignal that is under examination. This can be done by means of biosignal feedback. For example, when stimulating the brain with TMS, EEG signals are elicited and can be measured both in the stimulated brain region and in other brain regions that are electrically connected through neurons to the stimulated region. The observed EEG changes are often dependent on the state of the stimulated brain area and of the connected areas. Therefore, feedback EEG would provide a means for stabilizing the examination results. Among other suitable biosignals are also limb or finger movement sensors and muscle force measurements. In this context, discussion is concentrated in EMG measurements because it is the most probable application while bearing in mind that any measurable biosignal monitoring would be complementary with respect to the scope of the invention.

Many current applications for stimulating the motor cortex require the simultaneous use of both TMS and EMG. In some applications it is necessary to locate the primary motor areas of the brain by moving the stimulator coil in different locations and simultaneously observing the evoked EMG responses (see, for instance, Thickbroom G W, Mastaglia F L: Mapping Studies. In Handbook of Transcranial Magnetic Stimulation, 127-140, 2002, Arnold Publishers). The location giving the strongest EMG reflects the location of the primary motor cortex for the given muscle. Precise mapping requires also stereotactic coil localization (Krings T et al.: Introducing navigated transcranial magnetic stimulation as a refined brain mapping methodology. Neurosurg Rev. 2001:171-9; Ruohonen J: Background physics for magnetic stimulation. In: Transcranial Magnetic Stimulation and Transcranial Direct Current Stimulation. 1-14, 2003, Elsevier.). Additionally, it is necessary to determine the adequate stimulation intensity needed to elicit EMG responses. In most applications, this is done by varying the stimulation intensity while searching for an intensity that elicits EMG responses to 50% of the provided stimuli (Rossini P M et al. Non-invasive electrical and magnetic stimulation of brain, spinal cord and roots: Basic principles and procedures for routine clinical application. Report of an IFCN committee. Electroencephalography and Neurophysiology 1994; 91:79-92). For instance, depression treatment stimulation intensity is typically 80% of the motor threshold intensity. Advanced algorithms for the search of the motor threshold stimulation intensity have been proposed in the literature (e.g., Awiszus F; TMS and threshold hunting. Suppl Clin Neurophysiol. 2003; 13-23.)

The state of the stimulated brain region as well as that of the target muscles affect the results of determining the effects of motor cortex stimulation effects. For example, the motor threshold intensity greatly depends on the existing activity in the motor cortex and existing contraction of the muscle at the time of the TMS pulse. Existing muscle tone reduces the threshold and can thereby lead to poorly or even wrongly estimated stimulation intensity required in treatment trials or diagnostic examinations. Other applications use TMS evoked EMG responses to evaluate the functioning of the descending motor pathways. A delayed or otherwise abnormal evoked EMG is a sign of disease or trauma. Other applications include therapeutic uses where TMS is applied in trains over various parts of the brain: in treating depression over the prefrontal areas, and in treating pain over motoric areas.

Literature teaches the users to use EMG over the target muscles and to visually observe that TMS is performed when the target muscle is at rest. Publication U.S. Pat. No. 4,940,453 discloses a method for connecting electrical and magnetic stimulators together with an evoked potential recorder and analyzer. According to the method evoked responses triggered by a TMS pulse are recorded, but the evoked responses are not analyzed.

In some applications, it is more preferable to apply TMS examination so that the pulses are given when the muscle is already active, and that this activation is within predefined limits. The size and characteristics of the TMS-evoked response will be different depending on the level of the existing muscle tone; hence, better results are obtained when the TMS pulses are given at time points when the existing muscle tone is stabilized within predefined limits. Such applications measure the so-called active motor threshold, which is the intensity of TMS pulses required to elicit responses in pre-activated muscle. Some uses include measurement of the so-called silent period, which is a 50 to 300 ms duration of EMG silence in the pre-activated muscle activity followed by TMS pulse targeted to the cortical representation area of the same muscle (Oxford Handbook of Transcranial Stimulation, edited by E Wassermann et al., 2008). Determining whether or not the muscles are active at a level required and instructed by the operator is currently done with sensory aids. For example, the EMG signal is fed through an amplifier to loudspeakers and to the recipient's ears. Other solutions are visual indicators or number values shown to the operator.

However, both aural and visual aids for determining the correct activity level of the muscles are not ideally suitable for an environment in which TMS is typically applied. The need to observe a screen showing EMG values or to wait for an audible cue is considered burdensome by the operators. Furthermore, the human reaction time of an operator is not fast enough to observe a deviation from the EMG activity level exactly at the time of the TMS pulse, which lasts only some hundred microseconds and elicits muscle responses in 10 to 40 milliseconds. This tends to gratuitously lengthen the duration of each examination and leads to excessive analysis time after the examination to exclude those trials where the examination included unwanted levels of pre-TMS muscular contraction. Another immediate disadvantage is the increased number of needed stimulation pulses, also increasing the duration of the examinations. These short delays add up to a considerable amount of expensive operator time, e.g., over the life span of a TMS apparatus. For these reasons, practical subject examinations often therefore include trials recorded in different subject conditions, which reduces the usefulness of results of such examinations. On the whole, a major disadvantage of the known TMS apparatuses is their poor usability and dependency on human intentness.

### SUMMARY OF THE INVENTION

The invention is based on a new type of a magnetic stimulation method and apparatus for magnetic stimulation. The novel method comprises the steps of first determining a desired biosignal value or a range of values for at least one target, such as a muscle, and applying magnetic field pulses of short duration to the brain, and measuring the elicited biosignal value of each target before each magnetic field pulse. The method also comprises the step of preventing automatically the firing of a magnetic field pulse if the corresponding measured biosignal value is outside the predetermined limits.

More specifically, the method according to the invention is characterized by what is stated in the characterizing portion of the independent claim 1.

The invention is on the other hand based on a new type of an apparatus for magnetic stimulation comprising a TMS device including at least one coil and a trigger switch connected to the TMS device for the purpose of activating it. The apparatus also comprises a biosignal device, such as an electromyograph (EMG) device, which is adapted to monitor target activity values, such as the EMG values of a muscle, and a data processor, which is connected to the biosignal device and which linked to the TMS device. The apparatus further comprises means for processing the measured biosignal values into an analyzed number value, and means for preventing the firing of the TMS device if the analyzed number value is not within predetermined limits.

More specifically, the apparatus according to the invention is characterized by what is stated in the characterizing portion of the independent claim 12.

The invention also introduces a computer program product for a stimulation system, which is characterized by what is stated in the characterizing portion of claim 26.

Considerable advantages are gained with aid of the present invention. An immediate advantage is that any examination requiring the use of both TMS and EMG can be performed faster without human reaction time delays. Also, thanks to automated TMS trigger control, better ergonomics is achieved since the operator does not have to visually inspect the EMG screen while targeting and delivering the TMS pulses. A further benefit is that the results are more reliable and reproducible, because the status of muscle activity can be controlled and reproduced.

According to one embodiment of the invention, a further advantage is that the operator only needs to press the trigger switch of the TMS device and the system triggers the TMS pulse automatically immediately as soon as the target muscle is suitably active. This enhances the usability and user ergonomics as well as shortens the duration of TMS examinations.

According to another embodiment of the invention, a further advantage is gained by providing the system with a 3D localization tool, whose numeric data is used to ensure that a TMS pulse is administrated only when the TMS coil is in a correct position.

One benefit is that the invention eliminates the need for visual or auditory or other feedback to the operator, as the feedback from EMG can be used automatically by the computer system to control TMS pulse triggering. Another benefit is the reduction in the number of TMS pulses required to collect the necessary number of successfully elicited EMG responses. Yet another benefit is that time-demanding post-processing and analysis of the TMS trials are significantly reduced, since the operator does not need to browse all EMG responses to check the activity level preceding each TMS stimulus pulse.

In the following the invention is described with references to the accompanying drawings, in which:

### DESCRIPTION OF THE DRAWINGS

FIG. 1 shows a schematic overview of an environment in which TMS treatments are applied.

FIG. 2 shows a block diagram of a TMS arrangement according to prior art.

FIG. 3 shows a block diagram of a TMS arrangement according to one embodiment of the invention.

FIG. 4 shows a block diagram of a TMS arrangement according to another embodiment of the invention.

FIG. 5 shows the connections between TMS and EMG devices and a connecting computer.

### DESCRIPTION OF EXEMPLARY EMBODIMENTS

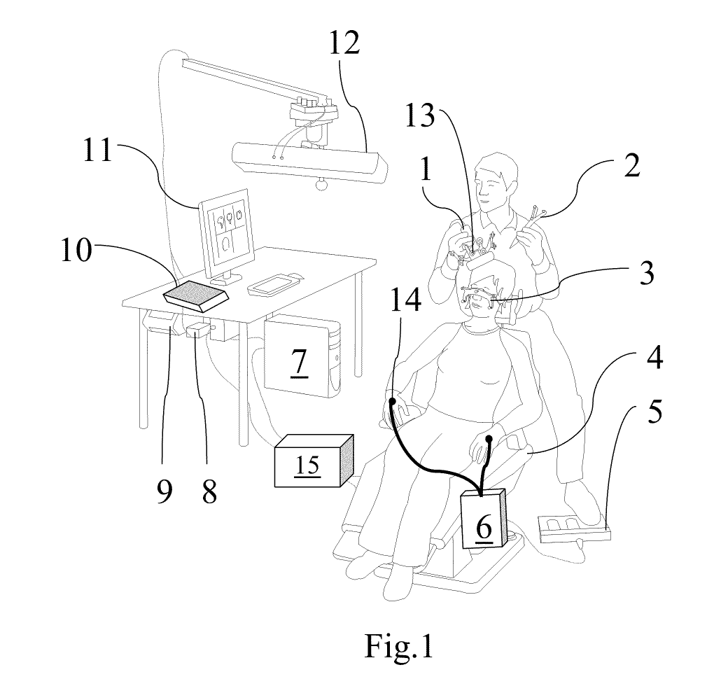

As illustrated in FIG. 1, the required equipment for stimulating the brain and measuring biosignals, such as EMG responses, according to the invention include a TMS device 15, and an EMG device, a data processor 7, i.e. an integrating computer 7 as well as auxiliary equipment such as cables and transformers 9. The EMG device comprises an EMG amplifier 6, a power supply 10 and electrodes 14. The patient is equipped with electrodes 14 of an EMG amplifier 6, which electrodes 14 are attached to the part of the patient being the object of interest, typically over the belly of one or more muscles. The EMG electrodes 14 record electrical potentials related to muscle activation. The recording of the signals can be time-locked to the TMS pulses related to record TMS evoked muscle responses. An EMG amplifier 6 is located adjacent to the patient chair 4 and amplifies the signal of the EMG electrodes 14. The biosignals are then digitized and fed to a processor or computer for display and analysis. The equipment can also detect other types of biosignals, such as EEG signals or muscle force responses, while EMG measurements are the most probable application. The EMG amplifier 6 is powered by an EMG power supply 10. The short TMS pulses are given with a TMS coil 1 for a duration of approximately 50 microseconds to 2 milliseconds, advantageously from 100 to 500 microseconds. Short pulses are more effective in stimulating the tissue, but there is typically a tradeoff between the electronics components and their costs and the realized pulse width. The TMS coil 1 is operated with a foot switch 5, i.e. a trigger switch, which triggers the given pulse. The foot switch 5 is connected to the TMS device 15, which fires a pulse through the TMS coil 1. The equipment further includes an integrating computer 7, which is here referred to as a controlling computer 7 and whose components as well as operating principle are discussed later on.

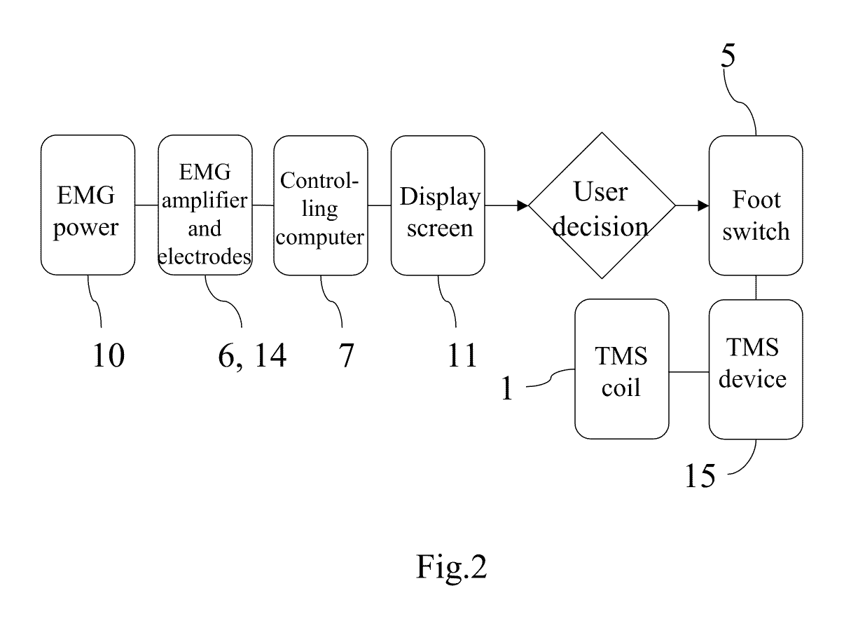

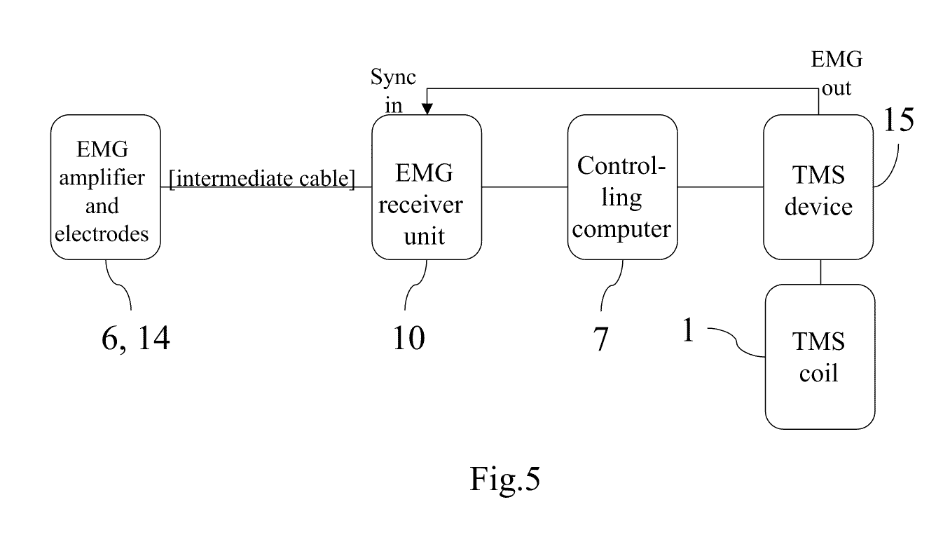

The arrangement of the prior art is illustrated as a block diagram in FIG. 2. As is apparent from the illustration, the major components of a TMS examination system are set up as separate entities so that the operation of the system as a whole is triggered by the firing of the TMS device 15 regardless of the activity level of the target muscle. This way the operator must ascertain that the pulses are fired when the target muscles are at a suitable activity level. However, the present invention is based on a novel electrical feedback between the EMG device and the TMS device 15, and the TMS coil 1 connected to the TMS device 15. Referring now to FIG. 5, the EMG signal is fed from the EMG amplifiers 6 to a signal processor unit (typically a controlling computer 7) via a USB cable, and the selected channel or channels are analyzed immediately. As is also apparent from FIG. 5, the EMG receiver unit 10, the controlling computer 7 and the TMS device 15 are linked together. The linkage can be provided by using, e.g. USB lines, wireless communication, or TTL level synchronization signals. When the operator presses the foot pedal or otherwise triggers a stimulus pulse, the control computer 7 sends a trigger signal to the TMS device 15. A synchronization signal is passed to the EMG device either directly by the computer or from the TMS device 15 so that the EMG signals can be related to the timing of the TMS pulse. The EMG can thereby be synchronized to the TMS pulses.

The TMS device 15 can be equipped with a means for localization of the coil with respect to individual brain's anatomical structures acquired using MR imaging. In this embodiment, the TMS coil is equipped with a coil tracker 13. The coil tracker 13 provides position information about the location and alignment of the TMS coil 1. A position sensor 12, located so that it is within unrestricted view of the trackers 3, 13, collects the position information of the head and coil trackers 3, 13 and is powered by a position sensor power supply unit 8. A preferable location for the position sensor 12 is the ceiling. A digitizer pen 2 is used for co-registering the live head with MR images of the same head. The computer then collects all localization information and can display to the user in real-time the exact location of the coil over the head and the stimulus distribution in the brain as an overlay on the MR images.

When a 3D localization system is used, there can be an additional signal to the control of the TMS triggering that controls the location of the coil with respect to the head. In studies that demand higher precision and repeatability, it is advantageous to have the coil at the same location during all TMS stimuli. Information from the 3D localization system can be used to decide whether a pulse is given or not, by determining whether the coil is in desired location and orientation. The limits vary with application. A typical limit could be less than 2-5 mm difference in the coil location and less than 5-10 degrees of difference in the coil's orientation.

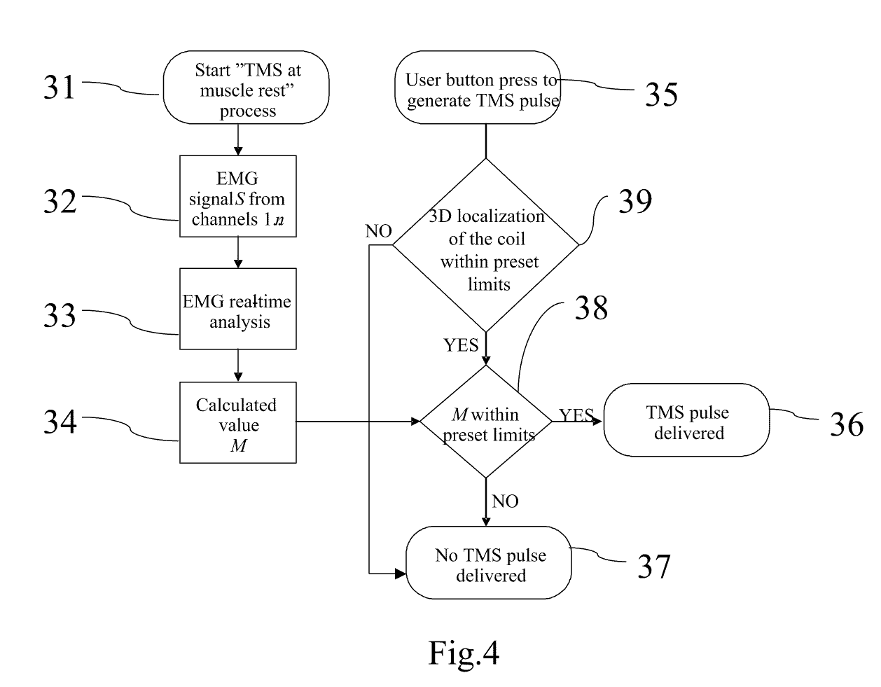

Referring now to FIGS. 1 and 4, according to another embodiment of the invention, the 3D localization system of the TMS equipment is used to provide additional information for controlling the administration of TMS pulses. The position information is preferably comparable numeric data. As illustrated in FIG. 4, the process is reinforced with an additional decision phase based on the position information of the 3D localization system. When preparing for a TMS treatment, apart from establishing limits for the patient's muscular activity, limits are also set for the position of the TMS coil 1. When the foot switch 5 is activated, the predetermined position limits are compared with real-time position information provided by the 3D localization system. The comparison can be performed in the control computer 7, for example, or in a separate calculating unit. If the coil 1 is in a correct position, i.e. position data is within the predetermined limits, the process can proceed in a conventional manner by checking if the calculated value M is within appropriate limits. If, however, the position information is not correct, i.e. the TMS coil 1 is misplaced, a TMS pulse is not administrated 39. Such an automated reassurance phase makes sure, that a pulse is given only when the TMS coil 1 is positioned correctly and only when the patient's muscular activity is within desirable limits.

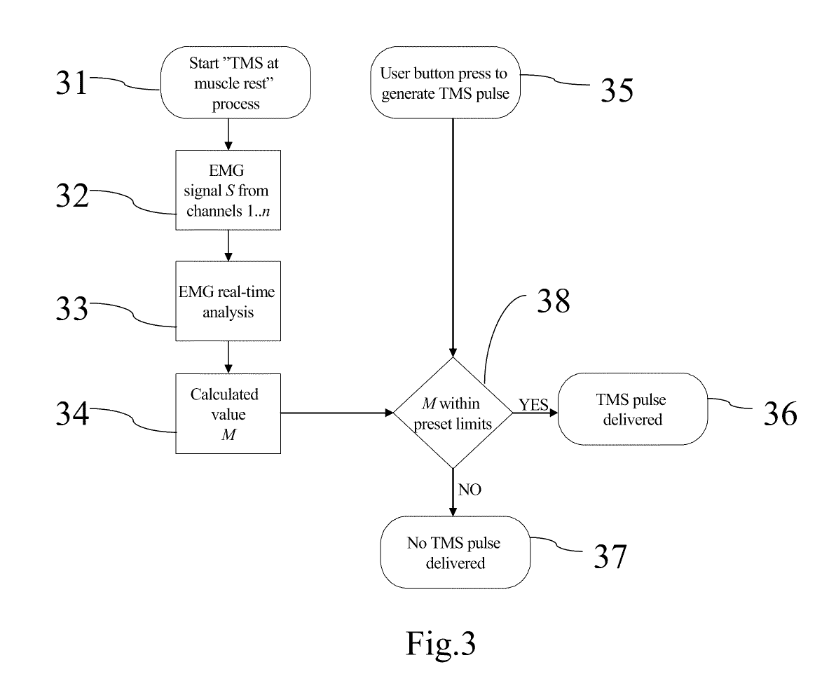

As illustrated in FIG. 3 and also referring to FIG. 1, the process includes a plurality of operations and one decision-making phase, which is done automatically. First, the operator switches the TMS coil 1 on 35 by pressing the foot switch 5, which starts the analyzing procedure 31. In the procedure EMG signals S are received 32 on channels 1 to n from the electrodes 1 to n of the EMG device. Next, the signals S are analyzed in real-time 33. It is paramount that the delay of the analyzing process 33 is as brief as possible so that the responsiveness of the whole system is not compromised. The analysis 33 of the EMG signals S can include, for instance, rectification of the signal and finding the peak signal, or computing continuously the moving area under the EMG signal curve acquired during short period of time, say 100 ms. The analysis results 34 in a single number value M.

Next, the single number value M is compared to a predetermined value or a range of values 38. If the number value M is within the predefined threshold values, a signal is transmitted to the software controlling the TMS stimulator 1, or directly to the TMS stimulator's hardware allowing the delivery of a TMS pulse 36. Otherwise the value comparison 38 results in a blocking signal. Generally speaking the blocking signal is transmitted, if the number value M is not within the preset limits. The signal then blocks firing of a TMS pulse 37 until the EMG activity is reduced below the threshold level. In mapping applications, it is also advantageous to perform the evaluation for several muscles simultaneously in several channels 1 to n.

In another form of the invention, the same analysis results are used to determine firing commands according to the activation level of the muscle. As in prior art, the number value M can be used to generate an audible or visual cue to the patient that helps the patient to reach and maintain or predefined muscular activity level. However, according to another embodiment of the invention, the software controlling the TMS stimulator can be advantageously set to fire a TMS pulse automatically when the correct activation level is reached besides blocking the firing when the activation level is outside the predefined limits. This way the operator only needs to hold the foot switch 5 pressed down and the system fires a TMS pulse immediately when a correct preset EMG value is reached.

Online evaluation of the EMG activity M can be based on different measures. Generally speaking, M can be any function of the S(i), where S(i) is the detected signal at time point i, and where the time point i is prior to the time point when the operator desires to fire a TMS pulse. N time points can be included that cover the length T of calculation window.

A possible measure is to first rectify the measured signal M, and then calculate the surface area below the rectified curve over a selected length of the recording. Such a measure would, for example, conform to an equation:

$$M = \frac{1}{T} {\sum }_{i = T}^{i = 0} \vert S ( i ) \vert Δ T ,$$

where
i=−T, T+1, . . . , −1, 0 denotes a sample of EMG data acquired,
i=0 is the most recent data point,
ΔT is the time between two data samples,
T is the length of the calculation window in time, and
S(i) is the detected signal at time point i. In case of surface EMG signals, it is the surface potential at time i.

This equation provides the average signal value M in a time window of T. An obvious extension is that S(i) is substituted by a manipulated signal derived from the actual recorded signal, such as manipulations by filtering or by mathematical functions like squared signal, square root, logarithms etc. It is advantageous to be able to adjust the length of the analysis window depending on the application. Also, it is advantageous to be able to add more recorded signals to the analysis equation.

Other possible analyzing equations include lightened versions, which do not take into account the time window T, for example. In the following, a couple of exemplary equations are listed as alternative analyzing tools:

$$M = \frac{1}{N} {\sum }_{i = N}^{i = 0} \vert S ( i ) \vert$$

$$M = \frac{1}{N} \sqrt{{\sum }_{i = 0}^{i = N} \vert S ( i ) \vert} ,$$

where
N is the number of samples during the analyzing period.

Accordingly, it is important that the result of the analysis produces a numeric value that is easy to compare with a predetermined value. This often requires taking an absolute value of the measured signal S to eliminate noise. The structure of the equation is therefore fairly optional as long as its product is easy to use.

A trigger signal to the TMS stimulator can be prevented when the calculated muscle tense in one or more muscles exceeds user-defined value. A trigger signal can be generated, if the muscle tense is between predetermined values. When evaluating whether or not the trigger signal should be generated, preconditions such as listed in the following conditions may, for example, be used:
1. Deliver pulses at rest only;
   if M>5 mV [symbol] prevent trigger signal to TMS.
2. Deliver pulses at user-defined activation level only;
   if M<4 mV or if M>5 mV [symbol] prevent trigger signal to TMS.

The analysis described above may be performed in any suitable device capable of producing the analyzed single number value M without any substantial delays. According to one embodiment of the invention, the analysis is performed in the data processing unit, i.e. the controlling computer 7 connected to the EMG device and linked to the TMS device 15. In other words, the means for analyzing the EMG values S into an analyzed number value M and means for preventing the firing of the TMS coil 1 if the analyzed number value M is not within predetermined limits is integrated into the software of the controlling computer 7. According to another embodiment, the analysis may be performed in a separate logic circuit connected to the EMG and TMS devices 15. The analysis may also be part of the hardware of the EMG device.

The resting state of a muscle can be determined as essentially zero EMG activity when recording with electrodes on the skin over the belly of the muscle. There is experimental and electrical noise present in the recorded and amplified EMG signal and after its digitization and hence the signal may be non-zero, although the muscle is completely at rest. In such case, the limiting value for judging that the muscle is at rest, is to be done on the basis of the internal noise in the amplifiers, device's filter settings, and on the basis of external electromagnetic noise present in the recording room coupling to the subject and the electronics. Typically the noise can be around 5-10 μV (rms). Activity of the adjacent muscles near the target muscle may also need to be taken into account when determining the threshold levels. Normally, however, the goal is that the pre-activity in the muscles is at least lower than 5% of the maximal activity. It is advantageous that the operator can adjust the threshold levels conveniently.

According to another embodiment of the invention, the controlling computer 7 may be equipped with a system, which gathers and displays information about the position and orientation of the TMS coil. These systems are stereotactic devices and they are typically based on emitting infrared radiation by means of the position sensor 12 and receiving the radiation reflected from the trackers 3, 13. Based on the emitted and received radiation patterns, the system concludes the position and orientation of the tool. This analysis can preferably be integrated to the controlling computer 7.

On the basis of the examples described above, it is obvious that within the scope of the invention, numerous solutions differing from the embodiments described above can be implemented. Furthermore, it is possible to gain a preferred embodiment of the invention by combining it with, for example, navigated TMS stimulation as disclosed in publication U.S. Pat. No. 6,8273,681. Thus the invention is not intended to be restricted to apply to only the examples described above, but instead the patent protection should be examined to the full extent of the accompanying claims.

TABLE 1
List of reference numbers

| Reference | |
|---|---|
| number | Part |
| 1 | TMS coil |
| 2 | Digitizer pen |
| 3 | Head tracker |
| 4 | Patient chair |
| 5 | Foot switch |
| 6 | EMG device (amplifier and electrodes) |
| 7 | Controlling computer |
| 8 | Position sensor power supply unit |
| 9 | Medical isolation transformer |
| 10 | EMG device power supply |
| 11 | Display |
| 12 | Position sensor |
| 13 | Coil tracker |
| 14 | Electrodes |
| 15 | TMS device |

1. A magnetic stimulation method comprising;
applying magnetic field pulses of short duration to the brain,
measuring a biosignal value for each intended target before each Transcranial Magnetic Stimulation (TMS) pulse,
determining first a desired limiting biosignal value, or a range of values, for at least one target, and
automatically preventing the firing of a TMS pulse if the corresponding measured biosignal value is outside the predetermined limit or limits.

2. A magnetic stimulation method according to claim 1, further comprising;
processing the measured biosignal value automatically and mathematically to form a single numeric value.

3. A magnetic stimulation method according to claim 2, further comprising;
making an absolute value of each measured biosignal value at a point of time out of a series of biosignal values measured at different points of time,
multiplying each absolute value of each measured biosignal value at a point of time by the time elapsed between two consecutive biosignal samples,
summing the series of the products,
comparing the product value to the predetermined limit values or values, and
controlling the magnetic field stimulation based on the results of the comparison.

4. A magnetic stimulation method according to claim 3, further comprising;
dividing the sum of the series by the length of the calculation time window resulting in the single number prior to its comparison with the predetermined limit values.

5. A magnetic stimulation method according to claim 1,
wherein automatically preventing the firing of a TMS pulse is performed if the biosignal value is not essentially zero.

6. A magnetic stimulation method according to claim 1,
wherein automatically preventing the firing of a TMS pulse is performed if the biosignal value is greater than a predetermined value of 50 mV.

7. A magnetic stimulation method according to claim 1,
wherein automatically preventing the firing of a TMS pulse is performed:
if the biosignal value is less than a predetermined lower limit value of 4 mV, or
if the biosignal value is greater than a predetermined upper limit value of 5 mV.

8. A magnetic stimulation method according to claim 1, further comprising;
transmitting an automatic trigger signal to a TMS device if the biosignal value is within predetermined limits.

9. A magnetic stimulation method according to claim 8, further comprising;
firing a TMS pulse automatically if a TMS trigger switch of the TMS device is turned on.

10. A magnetic stimulation method according to claim 1,
further comprising;
determining first a desired limiting position value, or a range of values, for a TMS coil;
monitoring a position value of the TMS coil with a 3D localization system, and
automatically preventing the firing of a TMS pulse if the corresponding measured position value of the TMS coil is outside the predetermined limit or limits.

11. A magnetic stimulation method according to claim 1,
further comprising;
monitoring biosignal value recordings simultaneously on a plurality of channels, and
combining the monitored values from the plurality of channels into a single numeric value.

12. An apparatus for magnetic stimulation comprising;
a Transcranial Magnetic Stimulation (TMS) device including at least one coil and a trigger switch for activation thereof,
a biosignal device adapted to monitor target activity values,
a data processor connected to the biosignal device,
wherein the data processor is linked to the TMS device;
means for processing the measured biosignal values into an analyzed number value, and
means for preventing the firing of the TMS device if an analyzed number value is not within predetermined limits.

13. An apparatus for a magnetic stimulation according to claim 12,
wherein the data processor is configured to:
process the biosignal values provided into an analyzed number value, and/or
prevent the firing of the TMS device if the analyzed number value is not within predetermined limits.

14. (canceled)

15. An apparatus for magnetic stimulation according to claim 12,
wherein the means for preventing the firing of the TMS device is adapted to feed a prevention signal to software controlling the TMS device.

16. An apparatus for magnetic stimulation according to claim 12,
wherein the means for preventing the firing of the TMS device is adapted to feed a prevention signal directly to hardware of the TMS device.

17. (canceled)

18. (canceled)

19. (canceled)

20. (canceled)

21. An apparatus for magnetic stimulation according to claim 12,
further comprising a means for transmitting a firing signal to the TMS device if an analyzed number value is within predetermined limits.

22. An apparatus for magnetic stimulation according to claim 21,
wherein the data processor is configured to transmit a firing signal to the TMS device if the analyzed number value is within predetermined limits.

23. An apparatus for magnetic stimulation according to claim 21.
wherein the TMS device is configured to fire a TMS pulse;
if it has received a firing signal, and
if the trigger switch is switched on.

24. An apparatus for magnetic stimulation according to claim 12,
further comprising
a 3D localization unit configured to monitor the position of the TMS coil and provide numeric position data, and
a means for preventing the firing of the TMS device if the position of the TMS device is not within predetermined limits.

25. An apparatus for magnetic stimulation according to claim 24,
wherein said means for preventing the firing of the TMS device is the data processor.

26. A computer program product stored on a non-transitory computer readable medium for causing a processor to perform the steps of,
causing magnetic field pulses of short duration to be applied to a brain by a Transcranial Magnetic Stimulation (TMS) device,
measuring a biosignal value for each intended target before each TMS pulse,
determining first a desired limiting biosignal value, or a range of values, for at least one target, and
automatically preventing the firing of a TMS pulse if the corresponding measured biosignal value is outside the predetermined limit or limits.
